(19) United States
(12) Patent Application Publication
(10) Pub. No.: US 2023/0043703 A1
(43) Pub. Date: Feb. 9, 2023

(54) DETECTION OF GENETIC VARIANTS

(71) Applicant: BIO-RAD LABORATORIES, INC., HERCULES, CA (US)

(72) Inventors: YAN WANG, SAN FRANCISCO, CA (US); STEVEN TAKASHI OKINO, SAN CARLOS, CA (US); JASON MA, HERCULES, CA (US)

(21) Appl. No.: 17/813,942

(22) Filed: Jul. 21, 2022

Related U.S. Application Data
(60) Provisional application No. 63/227,371, filed on Jul. 30, 2021.
(60) Provisional application No. 63/235,825, filed on Aug. 23, 2021.

(51) Int. Cl. C12Q 1/6827; C12Q 1/6883; C12Q 1/686

(52) U.S. Cl. CPC C12Q 1/6827; C12Q 1/6883; C12Q 1/686; C12Q 2600/156

(57) ABSTRACT

The subject invention pertains to the detection and differentiation of genetic variations by nucleic acid amplification. The invention provides methods of detecting one or more genetic variations in a nucleic acid that are in close proximity simultaneously. The invention further provides primer and probe oligonucleotides and methods of using said primers and probes in assays to detect genetic variants of concern of SARS-CoV-2. The methods of the invention detect genetic variants of other pathogens, including influenza, or genetic variants involved in inheritable diseases or cancer.

### CROSS-REFERENCE TO RELATED APPLICATIONS

This application claims the benefit of U.S. Provisional Application Ser. No. 63/227,371, filed Jul. 30, 2021, and 63/235,825, filed Aug. 23, 2021, the disclosures of which are hereby incorporated by reference in their entirety, including all figures, tables and amino acid or nucleic acid sequences.

### STATEMENT REGARDING SEQUENCE LISTING

The Sequence Listing for this application is labeled “Seq-List.xml” which was created on Jul. 10, 2022 and is 8,969 bytes. The entire content of the sequence listing is incorporated herein by reference in its entirety.

### BACKGROUND OF THE INVENTION

Nucleic acid detection is currently of a wide medical use, in particular in the field of virology and diagnosing and treating inheritable diseases. Indeed, viral identification in individuals suffering from viral diseases with variable genetic regions, such as SARS-CoV-2 or influenza, is essential to promoting human health.

Traditional methods of identifying the variations in the genetic code involve sequencing the nucleic acids; however, sequencing requires specialized instrumentation and is time consuming. Among the various known methods for nucleic acid identification, Polymerase Chain Reaction (PCR), particularly real-time PCR, is currently the most valued method, given both its sensitivity and its specificity. However, using PCR to identify the presence of nucleic acid sequence can lack the specificity required to discriminate between closely related genetic sequences in a single reaction.

Therefore, there remains a need for methods to efficiently detect genetic variants with enough specificity to distinguish related sequences without the use of genetic sequencing.

BRIEF SUMMARY OF THE INVENTION

The invention pertains to the detection of genetic variants of infectious agents and diseases. The invention provides primers and probes and methods of detecting by nucleic acid hybridization, specifically by nucleic acid amplification, more particularly, by PCR, advantageously by multiplex amplification (e.g., multiplex PCR), very advantageously, multiplex amplification of a single nucleic acid region with multiple variable genetic sites.

The invention also relates to methods of detecting infectious agents, specifically genetic variants of infectious agents. Additionally, genetic variations that can cause, or are associated with, diseases can be detected, particularly those diseases indicated by single nucleotide polymorphisms (SNPs).

The invention also relates to these primers and probes, as well as to pharmaceutical compositions, to biological compositions, to detection kits, and to diagnostic kits.

### BRIEF DESCRIPTION OF THE DRAWINGS

FIG. 1 shows a schematic depiction of a multiple variant assay targeting four sites in the SARS-CoV-2 spike gene that are mutated in SARS-CoV-2 variant strains. The MPC probe is a control that targets a conserved region in the SARS-CoV-2 spike gene.

FIGS. 2A-2E show the RT-qPCR assay traces of four different SARS-CoV-2 variant strains and the wild-type Wuhan strain of SARS-CoV-2 with an input of viral RNA at about 10,000 copies per reaction. The mutations identified by the multiple variant assay are consistent with the mutations present in each SARS-CoV-2 variant strain. This demonstrates that this technology can accurately identify mutations in RNA samples.

FIGS. 3A-3E show the RT-qPCR assay traces of four different SARS-CoV-2 variant strains and the wild-type Wuhan strain of SARS-CoV-2 with an input of viral RNA at about 0.5 copies per reaction. Not all reactions had RNA, the ones that do likely have only 1 or 2 RNA copies. Even with this trace RNA input, the mutations identified by the multiple variant assay are consistent with the mutations present in each SARS-CoV-2 variant strain. This demonstrates that this technology has high sensitivity and can accurately identify mutations in samples containing as little as a single RNA molecule. This implies, for example, that the individual variants can be identified from a highly diluted sample.

FIG. 4 shows the RT-qPCR assay traces of four different SARS-CoV-2 variant strains B.1.17 (United Kingdom), B.1.351 (South African), P.1 (Brazil), and B.1.429 (California), and the wild-type Wuhan strain of SARS-CoV-2 with an input of viral RNA at about 10,000 copies per reaction. Three amplicon sizes were tested: 335 bases with a forward primer with a 5′ base at 22,777 and a reverse primer with a 5′ base at 23,112; 860 bases with a forward primer with a 5′ base at 22,777 and a reverse primer with a 5′ base at 23,637; and 1,341 bases with a forward primer with a 5′ base at 21,771 and a reverse primer with a 5′ base at 23,112; base position reflects the SARS-CoV-2 genome coordinate based on the Wuhan strain. The mutations identified by the multiple variant assay are consistent with the mutations present in each SARS-CoV-2 variant strain for all amplicon lengths. This demonstrates that this technology can accurately identify RNA mutations using PCR amplicons of at least 1,341 bases.

FIG. 5 shows a schematic depiction of a multiple variant assay targeting four sites in the SARS-CoV-2 spike gene that are mutated in SARS-CoV-2 variant strains. The MPC probe is a control that targets a conserved region in the SARS-CoV-2 spike gene.

FIGS. 6A-6F show the RT-qPCR assay traces of five different SARS-CoV-2 variant strains (B.1.17 (United Kingdom), B.1.351 (South African), P.1 (Brazil), and B.1.429 (California), and B.1.617.2 (India)) and the wild-type Wuhan strain of SARS-CoV-2 with an input of viral RNA at about 10,000 copies per reaction when analyzed with the multiple variant assay. The mutations identified by the multiple variant assays are consistent with the mutations present in each SARS-CoV-2 variant strain. This demonstrates that this technology can accurately identify mutations in RNA samples.

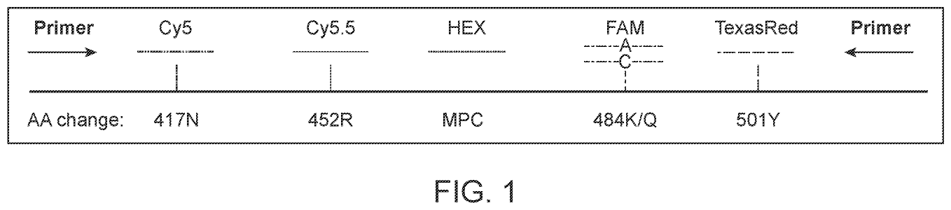

### BRIEF DESCRIPTION OF THE SEQUENCES

SEQ ID NO: 1: upstream primer that amplifies a portion of the receptor binding domain (RBD) of the SARS-CoV-2 spike protein.

SEQ ID NO: 2: downstream primer that amplifies a portion of the RBD of the SARS-CoV-2 spike protein.

SEQ ID NO: 3: fluorescent nucleotide probe with the 6-FAM fluorophore at the 5′ end and the IowaBlack FQ quencher at the 3′ end that detects a nucleotide mutation in the sequence encoding amino acid residue 484 of the SARS-CoV-2 spike protein.

SEQ ID NO: 4: fluorescent nucleotide probe with the HEX fluorophore at the 5′ end and the IowaBlack FQ quencher at the 3′ end that detects a constant region of the SARS-CoV-2 spike protein.

SEQ ID NO: 5: fluorescent nucleotide probe with the Texas Red-X fluorophore at the 5′ end and the IowaBlack RQ quencher at the 3′ end that detects a nucleotide mutation in the sequence encoding amino acid residue 501 of the SARS-CoV-2 spike protein.

SEQ ID NO: 6: fluorescent nucleotide probe with the ATTO 647 N fluorophore at the 5′ end and the IowaBlack RQ quencher at the 3′ end that detects a nucleotide mutation in the sequence encoding amino acid residue 417 of the SARS-CoV-2 spike protein.

SEQ ID NO: 7: fluorescent nucleotide probe with the Cy5.5 fluorophore at the 5′ end and the IowaBlack RQ quencher at the 3′ end that detects a nucleotide mutation in the sequence encoding amino acid residue 452 of the SARS-CoV-2 spike protein.

SEQ ID NO: 8: fluorescent nucleotide probe with the FAM fluorophore at the 5′ end that detects a nucleotide mutation in the sequence encoding amino acid residue 478 of the SARS-CoV-2 spike protein.

SEQ ID NO: 9: fluorescent nucleotide probe with the Cy5.5 fluorophore at the 5′ end that detects a nucleotide mutation in the sequence encoding amino acid residue 452 of the SARS-CoV-2 spike protein.

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. In the context of reagent and/or analyte concentrations, the term “about” can mean a range of up to 0-20%, 0 to 10%, 0 to 5%, or up to 1% of a given value. In the context of pH measurements, the terms “about” or “approximately” permit a variation of ±0.1 unit from a stated value.

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values.

The terms “label,” “detectable label,” “detectable moiety,” and like terms refer to a composition detectable by spectroscopic, photochemical, biochemical, immunochemical, chemical, or other physical means. For example, useful labels include fluorescent dyes (fluorophores), luminescent agents, electron-dense reagents, enzymes (e.g., as commonly used in an ELISA), biotin, enzymes acting on a substrate (e.g., horseradish peroxidase), digoxigenin, ³²P and other isotopes, haptens, and proteins which can be made detectable, e.g., by incorporating a radiolabel into the peptide or used to detect antibodies specifically reactive with the peptide. The term includes combinations of single labeling agents, e.g., a combination of fluorophores that provides a unique detectable signature, e.g., at a particular wavelength or combination of wavelengths. In the context of detecting nucleic acids (e.g., target sequences), the probes can, typically, be labeled with radioisotopes, fluorescent labels (fluorophores), or luminescent agents.

As used herein, the term “positive,” when referring to a result or signal, indicates the presence of an analyte or item that is being detected in a sample. The term “negative,” when referring to a result or signal, indicates the absence of an analyte or item that is being detected in a sample. Positive and negative are typically determined by comparison to at least one control, e.g., a threshold level that is required for a sample to be determined positive, or a negative control (e.g., a known blank). A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters, and will understand which controls are valuable in a given situation and be able to analyze data based on comparisons to control values. Controls are also valuable for determining the significance of data. For example, if values for a given parameter are variable in controls, variation in test samples will not be considered as significant.

As used herein, a “calibration control” is similar to a positive control, in that it includes a known amount of a known analyte. In the case of a real-time PCR assay, the calibration control can be designed to include known amounts of multiple known analytes. The amount of analyte(s) in the calibration control can be set at a minimum cut-off amount, e.g., so that a higher amount will be considered “positive” for the analyte(s), while a lower amount will be considered “negative” for the analyte(s). In some cases, multilevel calibration controls can be used, so that a range of analyte amounts can be more accurately determined. For example, an assay can include calibration controls at known low and high amounts, or known minimal, intermediate, and maximal amounts.

As used herein, “subject,” “patient,” “individual” and grammatical equivalents thereof are used interchangeably and refer to, except where indicated, mammals, such as humans and non-human primates, as well as rabbits, felines, canines, rats, mice, squirrels, goats, pigs, deer, and other mammalian species. The term does not necessarily indicate that the subject has been diagnosed with a particular disease, but typically refers to an individual under medical or veterinary supervision. A patient can be an individual that is seeking treatment, monitoring, adjustment or modification of an existing therapeutic regimen, etc.

The term “biological sample” or “sample from a subject” encompasses a variety of sample types obtained from an organism. The term encompasses bodily fluids such as blood, blood components, saliva, nasal mucous, serum, plasma, cerebrospinal fluid (CSF), urine and other liquid samples of biological origin, solid tissue biopsy, tissue cultures, or supernatant taken from cultured patient cells. In the context of the present disclosure, the biological sample is typically a bodily fluid with detectable amounts of antibodies or virus or with detectable amounts of a subject's genome, e.g., a tissue sample, blood or a blood component (e.g., plasma or serum), saliva, oropharyngeal, nasopharyngeal, or a nasal secretion (mucous). The biological sample can be processed prior to assay, e.g., to remove cells or cellular debris. The term encompasses samples that have been manipulated after their procurement, such as by treatment with reagents, solubilization, sedimentation, or enrichment for certain components.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

As used herein, the term “isolated nucleic acid” molecule refers to a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

As used herein, the term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

As used herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a nucleotide probe used in the method of this invention has at least 70% sequence identity, preferably 80%, 85%, 90%, 91%, 92%, 93%, 94%, 95%, 96%, 97%, 98%, 99%, or 100% identity, to a target sequence or complementary sequence thereof), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence.

### Probe and Primer Design and Detection

In certain embodiments, one or more pairs of primers that amplify a target nucleic acid (sometimes also referred to as target nucleotide sequence) region (or amplicon) of about 60 bp to about 5000 bp, about 60 bp to about 4000 bp, about 60 bp to about 3000 bp, about 60 bp to about 2000 bp, about 60 bp to about 1000 bp, about 60 bp to about 750 bp, about 60 bp to about 500 bp, about 100 bp to about 1000 bp, about 100 bp to about 500 bp, or about 300 to about 500 bp is provided by the subject invention. The primers for the amplification reactions can be designed according to known algorithms or by a skilled artisan. For example, algorithms implemented in commercially available or custom software can be used to design primers for amplifying the target sequences based on the complementarity and stringency of said primers to the target region. Stringency refers to hybridization conditions chosen to optimize binding of polynucleotide sequences with different degrees of complementarity. Stringency is affected by factors such as temperature, salt conditions, the presence of organic solvents in the hybridization mixtures, and the lengths and base compositions of the sequences to be hybridized and the extent of base mismatching, and the combination of parameters is more important than the absolute measure of any one factor.

Typically, the primers can be at least 12 bases, more often about 15, about 18, about 20, about 21, about 22, about 23, about 24, about 25, or about 30 base pairs in length. Primers are typically designed so that all primers participating in a particular reaction have melting temperatures that are within 5° C., and most preferably within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from about 0.1 μM to about 1 μM, about 0.2 μM to about 0.8 μM, about 0.3 μM to about 0.7 μM, about 0.4 μM to about 0.6 μM, or about 0.4 μM to about 0.5 μM.

In preferred embodiments, a single nucleotide amplification reaction of the subject invention contains one pair of primers (upstream and downstream primers) that amplifies a single nucleotide sequence. In certain embodiments, the primer pair can be SEQ ID NO: 1 and SEQ ID NO: 2 (Table 1), which amplifies a portion of the RBD domain of the spike protein of SARS-CoV-2.

TABLE 1

| | | | Size | |
|---|---|---|---|---|
| Oligo name | Fluorophore | Quencher | (bp) | Sequences (5′-3′) |
| 417F2 | none | None | 25 | TCATTTGTAATTAGAGGTGA |
| (SEQ ID NO: 1) | | | | TGAAG |
| 501R2 | none | None | 25 | AGTTCAAAAGAAAGTACTA |
| (SEQ ID NO: 2) | | | | CTACTC |
| SO484Q/K_FAM | 56-FAM | 3IABkFQ | 25 | CTTGTAATGGTGTTMAAGG |
| (SEQ ID NO: 3) | | | | TTTTAA |
| MPC2_HEX | 5HEX | 3IABkFQ | 25 | CCATATGATTGTAAAGGAAA |
| (SEQ ID NO: 4) | | | | GTAAC |
| 501Y_TRed | 5TexRd-XN | 3IAbRQSp | 18 | CCAACCCACTTATGGTGT |
| (SEQ ID NO: 5) | | | | |
| 417N_ATTN647 | 5ATTO647NN | 3IAbRQSp | 22 | CAAACTGGAAATATTGCTG |
| (SEQ ID NO: 6) | | | | ATT |
| 452R_Cy5.5 | 5Cy55 | 3IAbRQSp | 23 | ATTACCGGTATAGATTGTTT |
| (SEQ ID NO: 7) | | | | AGG |

TABLE 2

| Oligo name | Fluorophore | Size (bp) | Sequences (5′-3′) |
|---|---|---|---|
| Forward Primer | none | 25 | TCATTTGTAATTAGAGGTGA |
| (SEQ ID NO: 1) | | | TGAAG |
| Reverse Primer | none | 25 | AGTTCAAAAGAAAGTACTA |
| (SEQ ID NO: 2) | | | CTACTC |
| 478K Probe (SEQ | FAM | 18 | CCGGTAGCAAACCTTGTA |
| ID NO: 8) | | | |
| MPC Probe (SEQ | HEX | 25 | CCATATGATTGTAAAGGAAAGT |
| ID NO: 4) | | | AAC |
| 501Y Probe | Texas Red | 18 | CCAACCCACTTATGGTGT |
| (SEQ ID NO: 5) | | | |
| 417T Probe (SEQ | ATTO647 | 18 | TCAGCAATCGTTCCAGTT |
| ID NO: 9) | | | |
| 452R Probe (SEQ | 5Cy55 | 23 | ATTACCGGTATAGATTGTTT |
| ID NO: 7) | | | AGG |

In certain embodiments, probes can be designed to hybridize to a nucleic acid sequence, or portions thereof. In certain embodiments, the complementary nucleotide segment of the probe is 1, 2, 3, 4, 5, 6, 7, 8, 9, 10, 11, 12, 13, 14, 15, 16, 17, 18, 19, 20, 21, 22, 23, 24, 25, 30, 40, 50, or 100 base pairs long, or longer. In preferred embodiments, the complementary nucleotide segment of the probe is about 15 to about 60 base pairs, preferably about 16 to about 50 base pairs, more preferably about 17 to about 40 base pairs, more preferably about 17 to about 35 base pairs, more preferably about 18 to about 25 base pairs. In certain embodiments, at least 2, 3, 4, 5, 6, 7, 8, 9, 10, 15, 20, 25, 50, 75, 100, 125, or more probes can be used in a single reaction with multiple pairs of primers, or, preferably, a single pair of primers. Furthermore, the probe can be labeled with a fluorescent label (e.g., for use with a quencher label). The concentration of the probes can be optimized to promote the amplification reaction. In certain embodiments, the probes can be 100% complementary to a target sequence or at least 70%, 75%, 80%, 85%, 90%, 91%, 92%, 93%, 94%, 95%, 96%, 97%, 98%, or 99% sequence complementarity. In certain embodiments, the sequence of the probe can also have multiple possible alternative nucleotides represented by the IUPAC notation of, for example, R, Y, S, W, K, M, B, D, H, V, N, or a gap (“-” or “.”) nucleotide.

A number of formats are available that make use of fluorescent probes. These formats are often based on fluorescence resonance energy transfer (FRET) and include molecular beacon, Scorpion® probes, and TaqMan® probes. FRET is a distance-dependent interaction between a donor and acceptor molecule. The donor and acceptor molecules are fluorophores. If the fluorophores have excitation and emission spectra that overlap, then in close proximity (typically around 10-100 angstroms) the excitation of the donor fluorophore is transferred to the acceptor fluorophore. As a result, the lifetime of the donor molecule is decreased and its fluorescence is quenched, while the fluorescence intensity of the acceptor molecule is enhanced and depolarized. When the excited-state energy of the donor is transferred to a non-fluorophore acceptor, the fluorescence of the donor is quenched without subsequent emission of fluorescence by the acceptor. In this case, the acceptor functions as a quenching reagent.

One FRET-based format for real-time PCR uses DNA probes known as “molecular beacons” (see, e.g., Tyagi et al., Nat. Biotech. 16:49-53, 1998; U.S. Pat. No. 5,925,517). Molecular beacons have a hairpin structure wherein the quencher dye and reporter dye are in intimate contact with each other at the end of the stem of the hairpin. Upon hybridization with a complementary sequence, the loop of the hairpin structure becomes double stranded and forces the quencher and reporter dye apart, thus generating a fluorescent signal. A related detection method uses hairpin primers as the fluorogenic probe (Nazarenko et al., Nucl. Acid Res. 25:2516-2521, 1997; U.S. Pat. Nos. 5,866,336; 5,958,700). The PCR primers can be designed in such a manner that only when the primer adopts a linear structure, i.e., is incorporated into a PCR product, is a fluorescent signal generated. Amplification products can also be detected in solution using a fluorogenic 5′ nuclease assay, a TaqMan assay. See Holland et al., Proc. Natl. Acad. Sci. U.S.A. 88: 7276-7280, 1991; U.S. Pat. Nos. 5,538,848, 5,723,591, and 5,876,930. The TaqMan probe is designed to hybridize to a sequence within the desired PCR product. The 5′ end of the TaqMan probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. The excising of the 5′ fluorophore results in an increase in fluorescence which can be detected.

Schematically, said probe can have the following formulae: 5′ Fluorophore-probe-Quencher 3′ or 5′ Quencher-probe-Fluorophore 3′.

In addition to the hairpin and 5′-nuclease PCR assay, other formats have been developed that use the FRET mechanism. For example, single-stranded signal primers have been modified by linkage to two dyes to form a donor/acceptor dye pair in such a way that fluorescence of the first dye is quenched by the second dye. This signal primer contains a restriction site (U.S. Pat. No. 5,846,726) that allows the appropriate restriction enzyme to nick the primer when hybridized to a target. This cleavage separates the two dyes and a change in fluorescence is observed due to a decrease in quenching. Non-nucleotide linking reagents to couple oligonucleotides to ligands have also been described (U.S. Pat. No. 5,696,251).

Other fluorescent probes include inorganic molecules, multi-molecular mixtures of organic and/or inorganic molecules, crystals, heteropolymers, and the like. For example, CdSe—CdS core-shell nanocrystals enclosed in a silica shell may be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

In certain embodiments, multiplex PCR can be used in the subject methods. Multiplex PCR results in the detection of multiple polynucleotide fragments in the same reaction. See, e.g., PCR PRIMER, A LABORATORY MANUAL (Dieffenbach, ed. 1995) Cold Spring Harbor Press, pages 157-171. For instance, different probes that target different variable genetic sites can be added in parallel in the same reaction vessel. Multiplex assays can involve the use of different fluorescent labels to detect the different target sequences that are amplified. In preferred embodiments, a single pair of primers is used to amplify the target nucleotide sequence in order to degrade the fluorescent probes annealed to a sample nucleic acid sequence.

In certain embodiments, the probes herein can include any useful label, including fluorescent labels and quencher labels at any useful position in the nucleic acid sequence, such as, for example at the 3′- and/or 5′-terminus. Exemplary fluorescent labels include a quantum dot or a fluorophore. Examples of fluorescence labels for use in this method includes fluorescein, 6-FAM™ (Applied Biosystems, Carlsbad, Calif.), TET™ (Applied Biosystems, Carlsbad, Calif.), VIC™ (Applied Biosystems, Carlsbad, Calif), MAX, HEX™ (Applied Biosystems, Carlsbad, Calif.), TYE™ (ThermoFisher Scientific, Waltham, Mass.), TYE665, TYE705, TEX, JOE, Cy™ (Amersham Biosciences, Piscataway, N.J.) dyes (Cy2, Cy3, Cy3B, Cy3.5, Cy5, Cy5.5, Cy7), Texas Red® (Molecular Probes, Inc., Eugene, Oreg.), Texas Red-X, AlexaFluor® (Molecular Probes, Inc., Eugene, Oreg.) dyes (AlexaFluor 350, AlexaFluor 405, AlexaFluor 430, AlexaFluor 488, AlexaFluor 500, AlexaFluor 532, AlexaFluor 546, AlexaFluor 568, AlexaFluor 594, AlexaFluor 610, AlexaFluor 633, AlexaFluor 647, AlexaFluor 660, AlexaFluor 680, AlexaFluor 700, AlexaFluor 750), DyLight™ (ThermoFisher

Scientific, Waltham, Mass.) dyes (DyLight 350, DyLight 405, DyLight 488, DyLight 549, DyLight 594, DyLight 633, DyLight 649, DyLight 755), ATTO™ (ATTO-TEC GmbH, Siegen, Germany) dyes (ATTO 390, ATTO 425, ATTO 465, ATTO 488, ATTO 495, ATTO 520, ATTO 532, ATTO 550, ATTO 565, ATTO Rhol01, ATTO 590, ATTO 594, ATTO 610, ATTO 620, ATTO 633, ATTO 635, ATTO 637, ATTO 647, ATTO 647N, ATTO 655, ATTO 665, ATTO 680, ATTO 700, ATTO 725, ATTO 740), BODIPY® (Molecular Probes, Inc., Eugene, Oreg.) dyes (BODIPY FL, BODIPY R6G, BODIPY TMR, BOPDIPY 530/550, BODIPY 558/568, BODIPY 564/570, BODIPY 576/589, BODIPY 581/591, BODIPY 630/650, BODIPY 650/665), HiLyte Fluor™ (AnaSpec, Fremont, Calif.) dyes (HiLyte Fluor 488, HiLyte Fluor 555, HiLyte Fluor 594, HiLyte Fluor 647, HiLyte Fluor 680, HiLyte Fluor 750), AMCA, AMCA-S, Cascade® Blue (Molecular Probes, Inc., Eugene, Oreg.), Cascade Yellow, Coumarin, Hydroxycoumarin, Rhodamine Green™-X (Molecular Probes, Inc., Eugene, Oreg.), Rhodamine Red™-X (Molecular Probes, Inc., Eugene, Oreg.), Rhodamine 6G, TMR, ABY™ (Applied Biosystems, Carlsbad, Calif.), TAMRA™ (Applied Biosystems, Carlsbad, Calif.), 5-TAMRA, JUN™ (Applied Biosystems, Carlsbad, Calif.), ROX™ (Applied Biosystems, Carlsbad, Calif), Oregon Green® (Life Technologies, Grand Island, N.Y.), Oregon Green 500, IRDye® 700 (Li-Cor Biosciences, Lincoln, Nebr.), IRDye 800, WeIIRED D2, WeIIRED D3, WeIIRED D4, and Lightcycler® 640 (Roche Diagnostics GmbH, Mannheim, Germany). In some embodiments, bright fluorophores with extinction coefficients >50,000 M⁻¹ cm⁻¹ and appropriate spectral matching with the fluorescence detection channels can be used.

In certain embodiments, a fluorescently labeled probe is included in a reaction mixture and a fluorescently labeled reaction product is produced. Fluorophores used as labels to generate a fluorescently labeled probe included in embodiments of methods and compositions of the present invention can be any of numerous fluorophores including, but not limited to, 4-acetamido-4′-isothiocyanatostilbene-2,2′ disulfonic acid; acridine and derivatives such as acridine and acridine isothiocyanate; 4-amino-N-[3-vinylsulfonyl)phenyl]naphthalimide-3,5 disulfonate, Lucifer Yellow VS; N-(4-anilino-l-naphthyl)maleimide; anthranilamide, Brilliant Yellow; BIODIPY fluorophores (4,4-difluoro-4-bora-3a,4a-diaza-s-indacenes); coumarin and derivatives such as coumarin, 7-amino-4-methylcoumarin (AMC, Coumarin 120), 7-amino-4-trifluoromethylcoumarin (Coumarin 151); cyanosine; DAPDXYL sulfonyl chloride; 4′,6-diaminidino-2-phenylindole (DAPI); 5′,5″-dibromopyrogallol-sulfonephthalein (Bromopyrogallol Red); 7-diethylamino-3-(4′isothiocyanatophenyl)-4-methylcoumarin; dimethylenetriamine pentaacetate; 4,4′-diisothiocyanatodihydro-stilbene-2,2-disulfonic acid; 4,4′-diisothiocyanatostilbene-2,2′-disulfonic acid; 5-[dimethylamino]naphthalene-1-sulfonyl chloride (DNS, dansyl chloride); 4-4′-dimethylaminophenylazo)benzoic acid (DABCYL); 4-dimethylaminophenylazophenyl-4 sothiocyanate (DABITC); EDANS (5-[(2-aminoethyl)amino]naphthalene-1-sulfonic acid), eosin and derivatives such as eosin isothiocyanate; erythrosin and derivatives such as erythrosin B and erythrosin isothiocyanate; ethidium such as ethidium bromide; fluorescein and derivatives such as 5-carboxyfluorescein (FAM), hexachlorofluorescenin, 5-(4,6-dichlorotriazin-2-yl)aminofluorescein (DTAF), 2′,7′-dimethoxy-4′,5′-dichloro-6-carboxyfluorescein (JOE) and fluorescein isothiocyanate (FITC); fluorescamine; green fluorescent protein and derivatives such as EBFP, EBFP2, ECFP, and YFP; IAEDANS (5-({2-[(iodoacetyl)amino]ethyl}amino)naphthalene-1-sulfonic acid), Malachite Green isothiocyanate; 4-methylumbelliferone; orthocresolphthalein; nitrotyrosine; pararosaniline; Phenol Red; B-phycoerytnin; o-phthaldialdehyde; pyrene and derivatives such as pyrene butyrate, 1-pyrenesulfonyl chloride and succinimidyl 1-pyrene butyrate; QSY 7; QSY 9; Reactive Red 4 (Cibacron® Brilliant Red 3B-A); rhodamine and derivatives such as 6-carboxy-X-rhodamine (ROX), 6-carboxyrhodamine (Rhodamine 6G), rhodamine isothiocyanate, lissamine rhodamine B sulfonyl chloride, rhodamine B, rhodamine 123, sulforhodamine B, sulforhodamine 101 and sulfonyl chloride derivative of sulforhodamine 101 (Texas Red); N,N,N′,N-tetramethyl-carboxyrhodamine (TAMRA); tetramethyl rhodamine; tetramethyl rhodamine isothiocyanate (TRITC); riboflavin; rosolic acid; and terbium chelate derivatives. In certain embodiments, the concentration of the fluorescent probe in the compositions and method of use is about 0.01 μM to about 100 μM, about 0.1 μM to about 100 μM, about 0.1 μM to about 50 μM, about 0.1 μM to about 10 μM, or about 0.11 μM to about 1 μM. In certain embodiments, the concentration of the fluorescent probe is about 0.01 μM, about 0.1 μM, 0.2 μM, about 0.25 μM, about 0.3 μM, about 0.4 μM or about 0.5 μM.

Exemplary quencher labels include a fluorophore, a quantum dot, a metal nanoparticle, and other related labels. Suitable quenchers include Black Hole Quencher®-1 (Biosearch Technologies, Novato, Calif.), BHQ-2, Dabcyl, Iowa Black® FQ (Integrated DNA Technologies, Coralville, Iowa), IowaBlack RQ, QXL™ (AnaSpec, Fremont, Calif.), QSY 7, QSY 9, QSY 21, QSY 35, IRDye QC, BBQ-650, Atto 540Q, Atto 575Q, Atto 575Q, MGB 3′ CDPI3, and MGB-5′ CDPI3. In one instance, the term “quencher” refers to a substance which reduces emission from a fluorescent donor when in proximity to the donor. In preferred embodiments, the quencher is within 10, 11, 12, 13, 14, 15, 16, 17, 18, 19, 20, 21, 22, 23, 24, 25, 26, 27, 28, 29 or 30 nucleotide bases of the fluorescent label. Fluorescence is quenched when the fluorescence emitted from the fluorophore is detectably reduced, such as reduced by 10%, 20%, 30%, 40%, 50%, 60%, 70%, 80%, 90%, 95%, 99% or more.

In certain embodiments, each of the probes used in a single reaction can have a distinct fluorophore. In certain embodiments, the quencher of each probe can be identical or distinct. In specific embodiments, the probes can be SEQ ID NOs: 3-9. In specific embodiments, the fluorophore used for each probe can be 6-FAM, HEX, Texas Red-X, ATTO 647N, and Cy5.5, while the quenchers are IowaBlack FQ or IowaBlack RQ.

The invention further provides kits, including oligonucleotide probes and primers, packaged into suitable packaging material, optionally in combination with instructions for using the kit components, e.g., instructions for performing a method of the invention. In one embodiment, a kit includes an amount of an oligonucleotides probes and primers, and instructions for running the assay on a label or packaging insert. In further embodiments, a kit includes an article of manufacture, for performing the assay. Preferably, said kit comprises at one primer pair according to the invention. Said kit comprises more than one probe, e.g. at least two, at least three, at least four, at least five, at least six, at least 7, at least 8, at least 9, or at least 10 different probes, notably when the kit is intended to discriminate between different SARS-CoV-2 types or other infectious agents or genetic variations that cause disease.

In the kit according to the invention, the oligonucleotides (primers, probes) can be either kept separately, or partially mixed, or totally mixed.

Said oligonucleotides can be provided under dry form, or solubilized in a suitable solvent, as judged by the skilled person. Suitable solvents include TE, PCR-grade water, and the like.

In a preferred embodiment, the kit according to the invention can also contain further reagents suitable for a PCR or RT-PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes, including polymerases, such as Taq, Vent, Pfu (all of them Trade-Marks), activatable polymerase, reverse transcriptase, and the like; nucleotides like deoxynucleotides, dideoxunucleotides, dNTPs, dATP, dTTP, dCTP, dGTP, dUTP; other reagents, like DTT and/or RNase inhibitors; and polynucleotides like polyT, polydT, and other oligonucleotides, e.g., primers.

In another preferred embodiment, the kit according to the invention comprises PCR controls. Such controls are known in the art, and include qualitative controls, positive controls, negative controls, internal controls, quantitative controls, internal quantitative controls, as well as calibration ranges. The internal control for said PCR step can be a template which is unrelated to the target template in the PCR step. Such controls also may comprise control primers and/or control probes. For example, in the case of SARS-CoV-2 detection, it is possible to use as an internal control, a polynucleotide chosen within a gene whose presence is excluded in a sample originating from a human body (for example, from a plant gene), and whose size and GC content is equivalent to those from the target sequence. In other embodiments, a positive control is included which comprises a polynucleotide sequence associated with the target nucleotide sequence, such as an unmutated portion of the target nucleotide sequence (or amplicon). In some embodiments, the positive control is amplified by the oligonucleotide primer pair used to amplify the target nucleic acid sequence (for example, an unmutated portion of the SARS-CoV-2 spike protein sequence within an amplicon containing spike protein mutations within the RBD that is amplified by a pair of oligonucleotide primers). By way of example, positive control sequences for SARS-CoV-2 can be portions of the envelope, membrane, nucleocapsid gene, or invariant (unmutated) regions of the gene encoding the spike protein. For cells derived from specific human tissues, the positive control could be, for example, a portion of the following: the beta-actin gene, the aldolase gene, the dihydrofolate reductase gene, the glyceraldehyde phosphate dehydrogenase gene, the histone 3.3 gene, the hypoxanthine phosphoribosyltransferase gene, the Abelson gene (ABL), the BCR gene, the porphobilinogen deaminase gene (PBGD), or the beta-2-microglobulin gene (β2-MG).

In a preferred embodiment, the kit according to the invention contains means for extracting and/or purifying nucleic acid from a biological sample, e.g. from blood, serum, plasma, saliva, or nasal secretions. Such means are well known to those skilled in the art.

In a preferred embodiment, the kit according to the invention contains instructions for the use thereof. Said instructions can advantageously be a leaflet, a card, or the like. Said instructions can also be present under two forms: a detailed one, gathering exhaustive information about the kit and the use thereof, possibly also including literature data; and a quick-guide form or a memo, e.g., in the shape of a card, gathering the essential information needed for the use thereof. Instructions can therefore include instructions for practicing any of the methods of the invention described herein. For example, compositions can be included in a container, pack, or dispenser together with instructions for performing the nucleotide detection assay. Instructions may additionally include storage information, expiration date, or any information required by regulatory agencies such as the Food and Drug Administration or European Medicines Agency for use with a human or animal subject. The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, flash storage, electrical storage media such as RAM and ROM and hybrids of these such as magnetic/optical storage media.

In a preferred embodiment, said kit is a diagnostics kit, especially an in vitro diagnostics kit, i.e., an SARS-CoV-2 diagnostics kit.

The present invention also relates to the field of diagnostics, prognosis and drug/treatment efficiency monitoring, as above-described.

The oligonucleotides according to the present invention can be used for the identification of SARS-CoV-2 strains. In particular, the primers and probes according to the invention can be used for in vitro typing, sub-typing, and quantification of SARS-CoV-2 nucleic acids present in an in vitro sample, for instance, from a patient's nasal secretion sample.

### Nucleotide Detection

Any detection method or system able to detect a labelled nucleotide can be used in methods according to embodiments of the present invention and such appropriate detection methods and systems are well-known in the art. In certain embodiments, fluorescent microscopes, fluorescence scanners, spectrofluorometers and microplate readers, flow cytometers, or real-time PCR machine can be used to detect fluorescence.

In certain embodiments, the detection of the at least one single-stranded or double stranded nucleic acid is carried out in an enzyme-based nucleic acid amplification method.

The expression “enzyme-based nucleic acid amplification method” relates to any method wherein enzyme-catalyzed nucleic acid synthesis occurs.

Such an enzyme-based nucleic acid amplification method can be preferentially selected from the group constituted of LCR, Q-beta replication, NASBA, LLA (Linked Linear Amplification), TMA, 3 SR, Polymerase Chain Reaction (PCR), notably encompassing all PCR based methods known in the art, such as reverse transcriptase PCR (RT-PCR), simplex and multiplex PCR, real time PCR, end-point PCR, quantitative or qualitative PCR and combinations thereof. These enzyme-based nucleic acid amplification method are well known to the man skilled in the art and are notably described in Saiki et al. (1988) Science 239:487, EP 200 362 and EP 201 184 (PCR); Fahy et al. (1991) PCR Meth. Appl. 1:25-33 (3SR, Self-Sustained Sequence Replication); EP 329 822 (NASBA, Nucleic Acid Sequence-Based Amplification); U.S. Pat. No. 5,399,491 (TMA, Transcription Mediated Amplification), Walker et al. (1992) Proc. Natl. Acad. Sci. USA 89:392-396 (SDA, Strand Displacement Amplification); EP 0 320 308 (LCR, Ligase Chain Reaction); Bustin & Mueller (2005) Clin. Sci. (London) 109:365-379 (real-time Reverse-Transcription PCR).

In some embodiments, the enzyme-based nucleic acid amplification method is selected from the group consisting of Polymerase Chain Reaction (PCR) and Reverse-Transcriptase-PCR (RT-PCR), multiplex PCR or RT-PCR and real time PCR or RT-PCR. In other embodiments, the enzyme-based nucleic acid amplification method is a real time, optionally multiplex, PCR, quantitative PCR or RT-PCR method.

As intended herein “multiplex” relates to the detection of at least two different nucleic acid targets within a single 10 bp to 1000 bp target nucleotide sequence by using at least two oligonucleotide probes, wherein each one of said nucleic acid targets is detectable by at least one of said probes. Preferably, the labelling of each probe with a different fluorescent donor makes it possible to detect separately the signal emitted by the distinct probes bound to their target nucleic acid. In preferred embodiments, at least three oligonucleotide probes are used to detect of at least three different nucleic acid targets within a single 10 bp to 1000 bp target nucleotide sequence. In more preferred embodiments, at least four oligonucleotide probes are used to detect of at least four different nucleic acid targets within a single 10 bp to 1000 bp target nucleotide sequence. In even more preferred embodiments, at least five oligonucleotide probes are used to detect of at least five different nucleic acid targets within a single 10 bp to 1000 bp target nucleotide sequence. Typically, the target sequence contains two or more nucleotide mutations (genetic variations) that permit the probes to distinguish between the mutations found within the target sequences.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Successful PCR amplification requires high yield, high selectivity, and a controlled reaction rate at each step. Yield, selectivity, and reaction rate generally depend on the temperature, and optimal temperatures depend on the composition and length of the polynucleotide, enzymes and other components in the reaction system. In addition, different temperatures may be optimal for different steps. Optimal reaction conditions may vary, depending on the target sequence and the composition of the primer. Thermal cyclers such as, for example, real-time PCR systems provide the necessary control of reaction conditions to optimize the PCR process for a particular assay. For instance, a real-time PCR system may be programmed by selecting temperatures to be maintained, time durations for each cycle, number of cycles, and the like. In some embodiments, temperature gradients may be programmed so that different sample wells may be maintained at different temperatures, and so on.

In certain embodiments, the target nucleic acid sequence can be RNA or DNA. RNA or DNA can be artificially synthesized or isolated from natural sources. In some embodiments, the RNA target nucleic acid sequence can be a ribonucleic acid such as RNA, mRNA, piRNA, tRNA, rRNA, ncRNA, gRNA, shRNA, siRNA, snRNA, miRNA and snoRNA More preferably the DNA or RNA is biologically active or encodes a biologically active polypeptide. The DNA or RNA template can also be present in any useful amount.

Reverse transcriptases useful in the present invention can be any polymerase that exhibits reverse transcriptase activity. Preferred enzymes include those that exhibit reduced RNase H activity. Several reverse transcriptases are known in the art and are commercially available (e.g., from Bio-Rad Laboratories, Inc., Hercules, Calif.; Boehringer Mannheim Corp., Indianapolis, Ind.; Life Technologies, Inc., Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). In some embodiments, the reverse transcriptase can be Avian Myeloblastosis Virus reverse transcriptase (AMV-RT), Moloney Murine Leukemia Virus reverse transcriptase (M-MLV-RT), Human Immunovirus reverse transcriptase (HIV-RT), EIAV-RT, RAV2-RT, C. hydrogenoformans DNA Polymerase, rTth DNA polymerase, SUPERSCRIPT I, SUPERSCRIPT II, and mutants, variants and derivatives thereof. It is to be understood that a variety of reverse transcriptases can be used in the present invention, including reverse transcriptases not specifically disclosed above, without departing from the scope or preferred embodiments disclosed herein.

DNA polymerases useful in the present invention can be any polymerase capable of replicating a DNA molecule. Preferred DNA polymerases are thermostable polymerases and polymerases that have exonuclease activity, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothemophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Many DNA polymerases are known in the art and are commercially available (e.g., from Bio-Rad Laboratories, Inc., Hercules, Calif; Boehringer Mannheim Corp., Indianapolis, Ind.; Life Technologies, Inc., Rockville, Md; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif). In some embodiments, the DNA polymerase can be Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, and active mutants, variants and derivatives thereof. It is to be understood that a variety of DNA polymerases can be used in the present invention, including DNA polymerases not specifically disclosed above, without departing from the scope or preferred embodiments thereof.

The reverse transcriptase can be present in any appropriate ratio to the DNA polymerase. In some embodiments, the ratio of reverse transcriptase to DNA polymerase in unit activity is greater than or equal to 3. One of skill in the art will appreciate that other reverse transcriptase to DNA polymerase ratios are useful in the present invention.

In a preferred embodiment, the reactions according to the invention can also contain further reagents suitable for a PCR step.

Such reagents are known to those skilled in the art, and include water, like nuclease-free water, RNase free water, DNAse-free water, PCR-grade water; salts, like magnesium, magnesium chloride, potassium; buffers such as Tris; enzymes; nucleotides like deoxynucleotides, dideoxunucleotides, dNTPs, dATP, dTTP, dCTP, dGTP, dUTP and modified nucleotides such as deaza-, locked nucleic acid, and peptide nucleic acid; other reagents, like DTT and/or RNase inhibitors; and polynucleotides like polyT and polydT.

In preferred embodiments, the methods of the subject invention use the Reliance One-Step Multiplex RT-qPCR Supermix (Bio-Rad Laboratories, Inc.).

Certain method and compositions used for amplifying and/or detecting nucleic acids are described in U.S. Pat. Nos. 9,493,824, 10,988,762, 10,053,676, 6,627,424, 7,541,170, 7,666,645, 7,560,260, 8,367,376, 9,145,550, 9,688,969, 10,577,593, 10,301,675, 8,338,094, and 9,200,318, which are each entirely incorporated herein by reference. U.S. Pat. No. 9,493,824 describes nucleic acid amplification/detection reaction mixtures and uses thereof; U.S. Pat. No. 10,988,762 describes reverse transcriptases and uses thereof; U.S. Pat. No. 10,053,676 describes polymerase storage compositions and uses thereof; U.S. Pat. No. 6,627,424 describes DNA polymerases and uses thereof; U.S. Pat. No. 7,541,170 describes polymerases and uses thereof; U.S. Pat. No. 7,666,645 describes polymerases and uses thereof; U.S. Pat. No. 7,560,260 describes polymerases, particularly Pfu polymerases, and uses thereof; U.S. Pat. No. 8,367,376 describes polymerases, particularly Pfu polymerases, and uses thereof; U.S. Pat. No. 9,145,550 describes polymerases, particularly Pfu polymerases, and uses thereof; U.S. Pat. No. 9,688,969 describes polymerases, particularly Pfu polymerases, and uses thereof; U.S. Pat. No. 10,577,593 describes polymerases, particularly Pfu polymerases, and uses thereof; U.S. Pat. No. 10,301,675 describes compositions and methods for synthesizing cDNA from an RNA template and replicating the cDNA and kits thereof; U.S. Pat. No. 8,338,094 describes methods for synthesizing cDNA from an RNA template and replicating the cDNA and kits thereof; and U.S. Pat. No. 9,200,318 describes methods for synthesizing cDNA from an RNA template and replicating the cDNA and kits thereof.

### Targets of Nucleotide Detection

In certain embodiments, the methods provided by the subject invention can be used to detect one or more genetic variations or variants of infectious agents or in a subject's genome (also referred to as “target sequence(s)”, “target nucleic acid sequence(s)”, or “target nucleotide sequence(s)” herein). In certain embodiments, genetic variations (mutations) can be a substitution, addition, or deletion of at least 1, 2, 3, 4, 5, 6, 7, 8, 9, 10, 11, 12, 15, 18, 21, 25, 30, 35, 40, 50, 100, or more nucleotides, which can cause a substitution, addition, or deletion of at least 1, 2, 3, 4, 5, 6, 7, 8, 9, 10, 11, 12, 13, 14, 15, 16, 17, 18, 19, 20, 25, 30, 33 or more amino acids. In preferred embodiments, these one, two or more genetic variations are found within a target sequence that is between about 60 and about 5000 nucleotides in length.

In certain embodiments, the methods of the subject invention can be used to detect genetic variants of RNA viruses, such as, for example, Middle East respiratory syndrome-related coronavirus (MERS), severe acute respiratory syndrome coronavirus (SARS-CoV); SARS-CoV-2, including genetic variants, such as, for example, B.1.351 (South African), B.1.17 (United Kingdom), P.1 (Brazil), B.1.429 (California), B.1.617 (India), and B.1.617.2 (India) when compared to the wild-type Wuhan virus; influenza, including genetic variants of genes that can be used to differentiate between the 4 types of influenza (A, B, C, and D) and genetic variants of the genes encoding hemagglutinin (HA) and neuraminidase (NA); enteroviruses, including genetic variants of genes that can be used to differentiate between Enteroviruses A-L and serotypes of Enteroviruses A-L; human immunodeficiency virus (HIV), including genetic variants of genes that can be used to differentiate between the two groups (HIV-1 and HIV-2) and the various subtypes (clades), which can be differentiated by, for example, the envelop-encoding region; DNA viruses, including human papillomavirus (HPV), including genetic variants of genes, such as, E1, E2, E3, E4, E5, E6, E7, Ll, L2, and the Long Coding Region (LRC) of HPV that can be used differentiate common strains, such as, for example, HPV 16, 18, 31, 33, and 45, and other strains described in Munoz N, Bosch FX, de Sanjosé S, Herrero R, Castellsagué X, Shah K V, Snijders P J, Meijer C J; International Agency for Research on Cancer Multicenter Cervical Cancer Study Group. Epidemiologic classification of human papillomavirus types associated with cervical cancer. N Engl J Med. 2003 Feb 6;348(6):518-27. doi: 10.1056/NEJMoa021641, which is hereby incorporated by reference; single-nucleotide polymorphism (SNPs) that can cause diseases and can be used to identify variants of a disease, identify susceptibility of risks to disease, identify severity of a disease, and/or identify effectiveness of treatment of a disease. The disease can include, for example, sickle cell-anemia, β-thalassemia, cystic fibrosis, Alzheimer's disease, and cancer, including breast, lung, bladder, colon, and prostate cancers that each has genes in which genetic variants are associated with determining susceptibility risk to each cancer. The probes and primers of Tables 1 and 2 can be used to detect target nucleic acids obtained from SARS-CoV-2.

As discussed above, the disclosed multiplex methods are capable of identifying genetic variants or genetic variations in amplicons of between about 5 and about 1000 nucleotides. Various databases also exist that identify genes containing genetic variations that are identified with various diseases, such as human diseases and archived versions of the genes (including information available at the filing date of this application) associated with the diseases can be accessed using various public databases such as those available at the NCBI or Uniprot web sites. For example, the database available at uniprot.org/uniprot/?query=keyword:%22Disease%20 [KW-9995]%22&fil=organism%3 A%22Homo+sapiens+%28Human%29+%5B9606%5D%22+AND+reviewed%3Ayes identifies genes and the amino acid mutations that are associated with human diseases. Primers are used to amplify target sequences within these genes that contain two or more genetic variations (mutations) and that are between about 5 and about 1000 nucleotides in length. The target sequences that are produced can then be probed as disclosed herein (with multiple probes, each of which identify a specific mutation) to identify the genetic variations present within the gene that is associated with a particular human disease. Another source that identifies gene targets suitable for use in the disclosed methods is found at uniprot.org/docs/humsavar.txt (or at web.archive.org/web/20210215000000*/ uniprot.org/docs/humsavar.txt or web.archive.org/web/20210225195708/ uniprot.org/docs/humsavar.txt). This source identifies gene targets and provides accession numbers and amino acid changes present within the gene and their association with disease. Yet another source of clinically relevant variants is found in the ClinVar database that can be accessed at ncbi.nlm.nih.gov/clinvar/. This database is a public archive of clinically significant variants and is discussed in Landrum et al., Nucl. Acids. Res., 2016, 44:D862-868, the disclosure of which is hereby incorporated by reference in its entirety. Each of these databases are hereby incorporated by reference in their entireties.

### MATERIALS AND METHODS

### RNA Processing

If the sample is an RNA sample, the cell sample can be lysed or total RNA can be isolated. To prepare a lysate from cells, SingleShot Cell Lysis RT-qPCR kits (Bio-Rad Laboratories, Inc., Hercules, Calif.) is used. To isolate RNA from a sample, the Aurum Total RNA Mini Kit (Bio-Rad Laboratories, Inc.) or PureZOL RNA Isolation Reagent (Bio-Rad Laboratories, Inc.) can be used.

### RT-PCR Materials

The following components are used to perform a RT-PCR of the subject invention: Reliance One-Step RT-qPCR Multiplex Supermix (Bio-Rad Laboratories, Inc.), PCR plates, adhesive seals for the plates, primers for amplifying the genetic region (e.g., SEQ ID NO: 1 and SEQ ID NO: 2), fluorescent probes (e.g., SEQ ID NO: 3, SEQ ID NO: 4, SEQ ID NO: 5, SEQ ID NO: 6, SEQ ID NO: 7, SEQ ID NO: 8, and SEQ ID NO: 9) that bind to target variable genetic regions (e.g., spike protein of SARS-CoV-2). Additionally a real-time PCR Detection system is needed (e.g., CFX96 Touch or CFX384 Touch Real-Time PCR Detection System).

### RT-PCR Procedure

The Reliance One-Step RT-qPCR Multiplex Supermix is delivered in a 4× ready-to-use format. To use the mix, thaw the vial on ice to 4° C. Thoroughly mix the vial and briefly centrifuge to ensure all components are at the bottom of the tube. Store on ice protected from light until ready to use.

If using automated liquid handling, let sit at ambient temperature for 10 min to further reduce the viscosity of the Supermix.

The primers and probes are then prepared at 1× concentration and added to the Supermix, nuclease-free water, and RNA template in the PCR Plate and the plate is sealed. The compositions are then vortexed and centrifuged.

The Real-Time PCR Detection System can be programmed according to the following procedure:

1. Reverse Transcriptase at 50° C. for 10 minutes

2. DNA polymerase activation and template denaturation at 95° C. for 10 minutes

3. Amplification-Template denaturation at 95° C. for 10 seconds

4. Amplification-Annealing/Extension and plate read at 58° -60° C. for 30 seconds

5. Repeat steps 3 and 4 45-50 times.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1—Individual Genetic Variations of SARS-CoV-2 can be Distinguished in a Single PCR Reaction

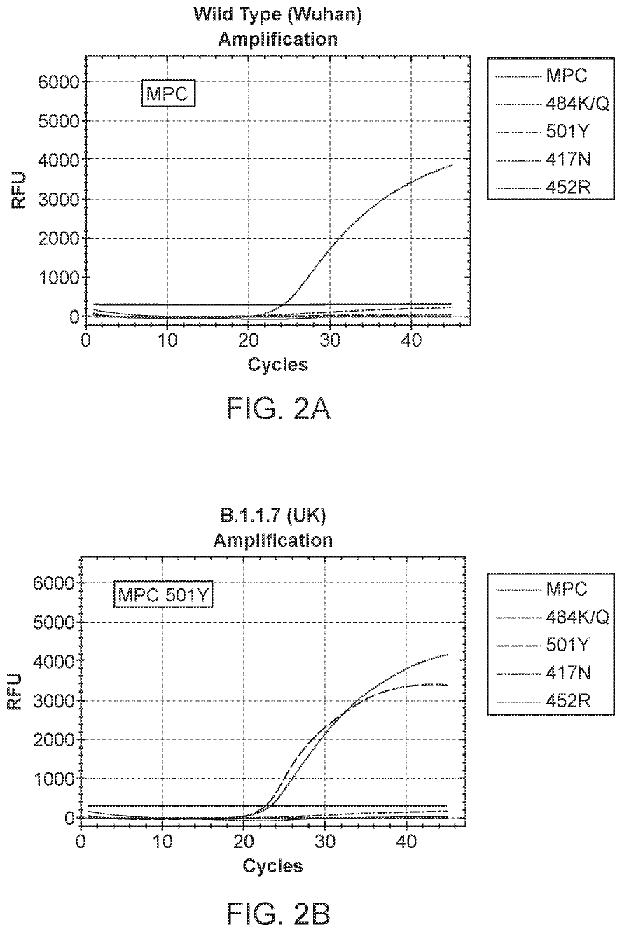

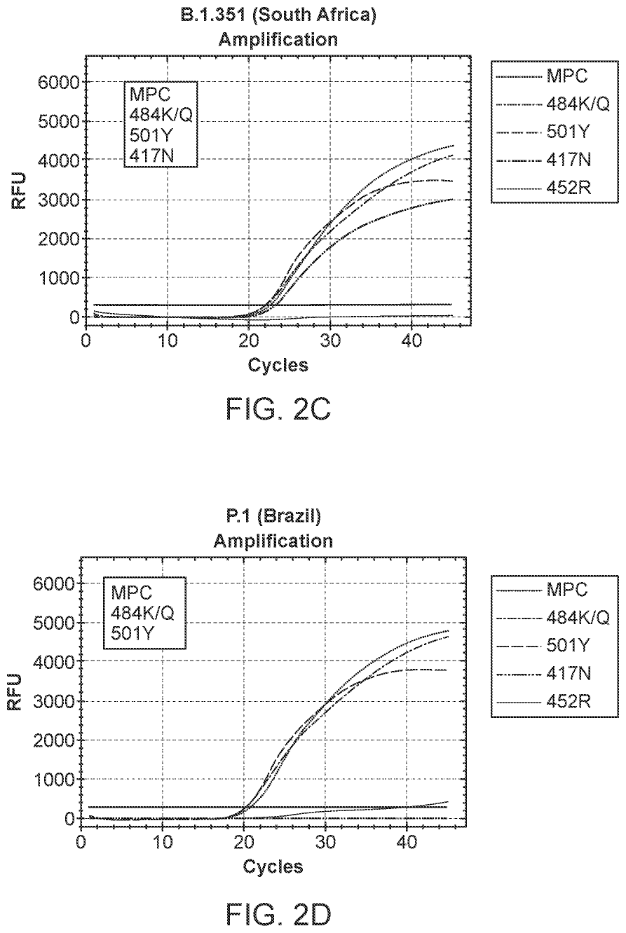

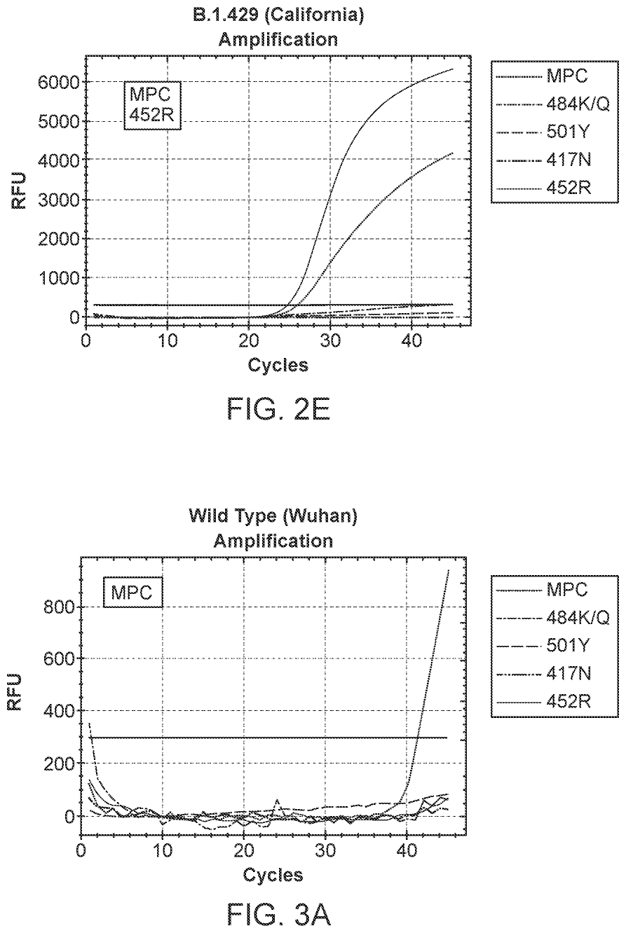

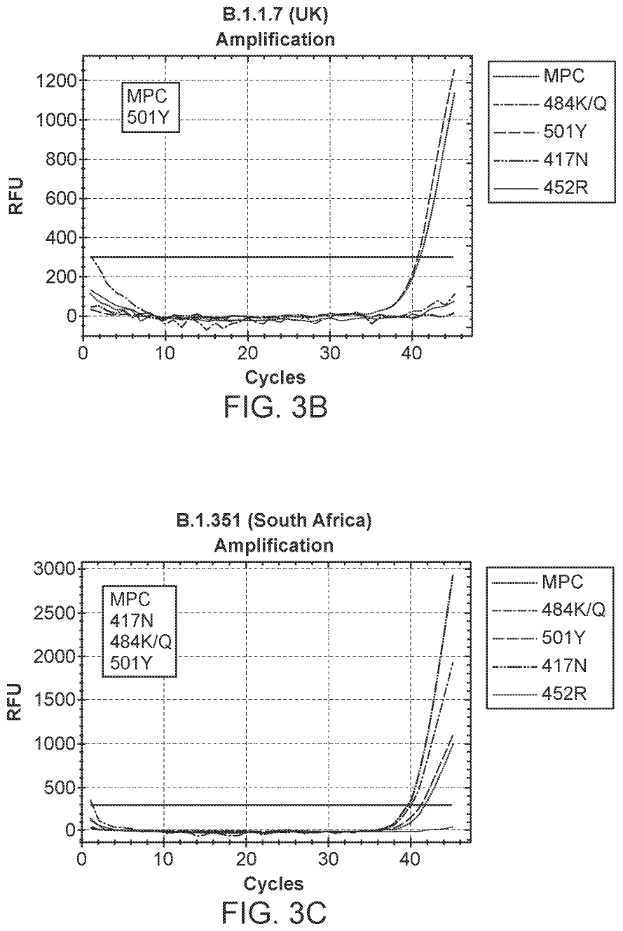

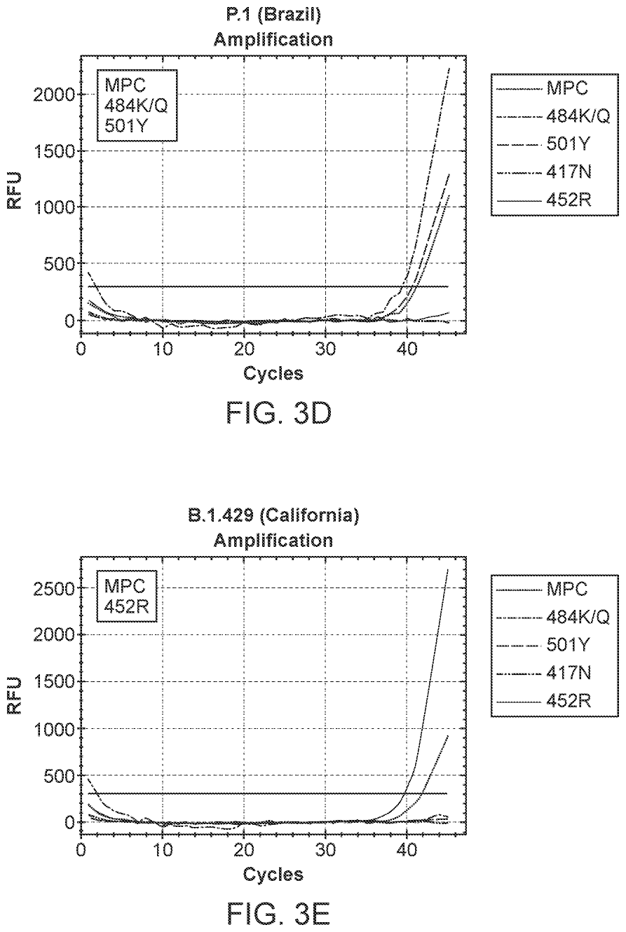

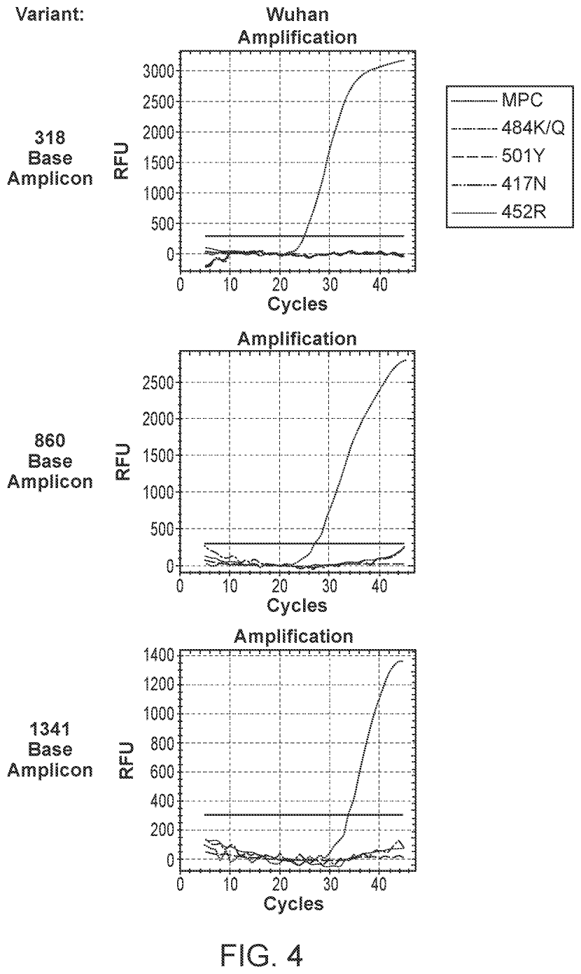

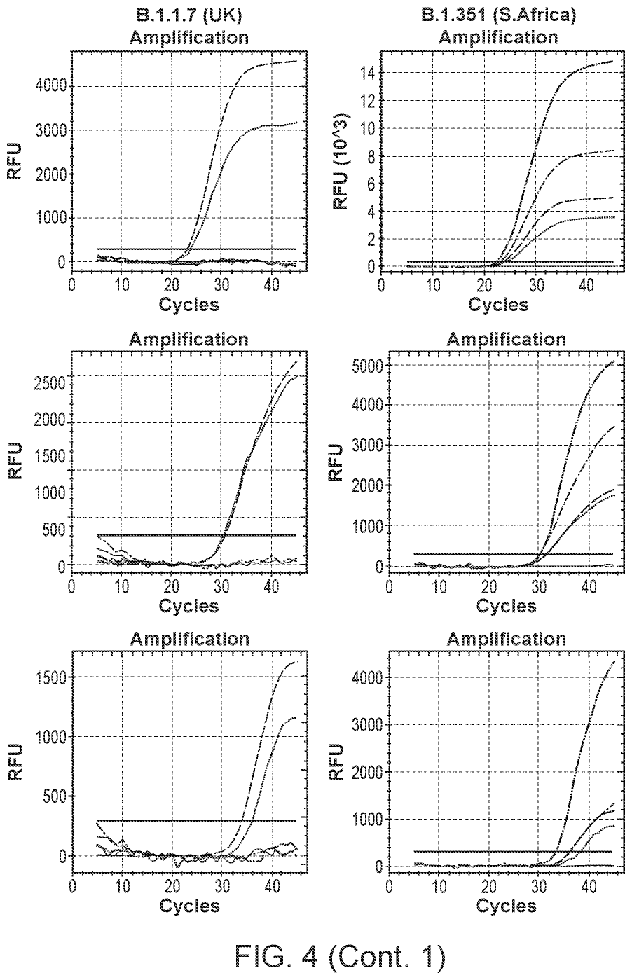

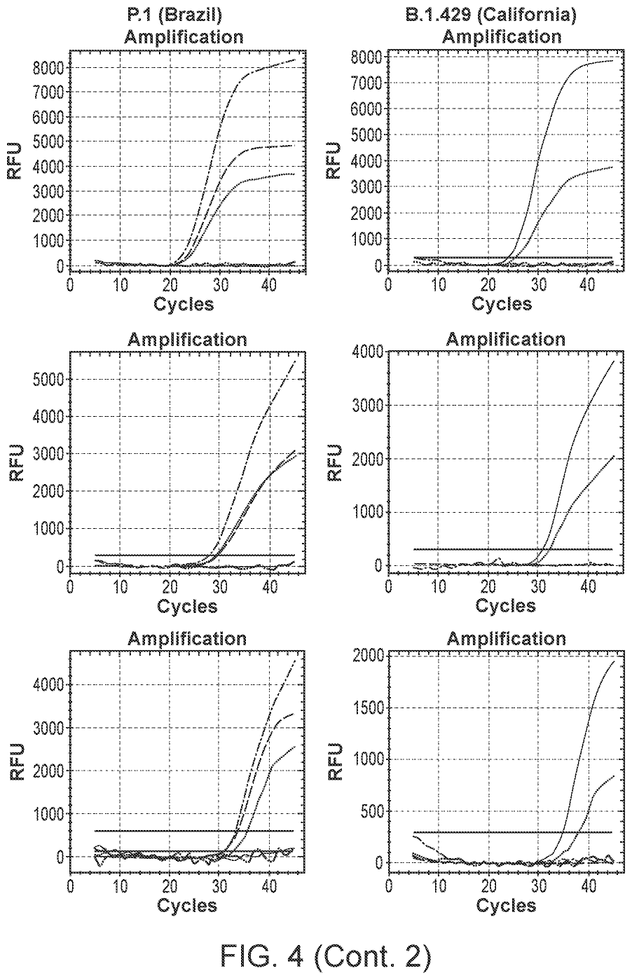

A forward and a reverse primer are designed to amplify of a 330 bp amplicon region that contains the known key mutations of the SARS-CoV-2 spike protein RBD region. Within this region, 5 dual-labeled probes are designed to detect (1) a highly conserved region that serves as the control (HEX channel), (2) the 417N mutation (Cy5 channel), (3) the 452R mutation (Cy5.5 channel), (4) the 484Q/K mutation (FAM channel), and (5) 501Y mutation (Texas Red) channel. The 484Q/K probe contains a degenerate nucleotide that is 50% A and 50% C to enable the detection of either the 484K or the 484Q mutation. The final reaction mix contains the two primers and the 5 probes added to the Reliance One-Step Multiplex Supermix and RNA extracted from respiratory patient samples. Positive amplification in the control channel (HEX) indicates that the samples contain SARS-CoV-2 virus. Depending on which mutation is amplified, the variant strain can be determined. The UK variant can be identified if the 501Y amplifies (FIG. 2B and FIG. 3B). The South Africa Variant can be identified if 501Y, 417N and 484Q/K amplify (FIG. 2C and FIG. 3C). The Brazil variant can be identified if 501Y and 484Q/K amplify (FIG. 2D and FIG. 3D). The California variant can be identified if 452R amplifies (FIG. 2E and FIG. 3E). If the sample contains the wild-type SARS-CoV-2, only the positive control is expected to be amplified (FIG. 2A and FIG. 3A).

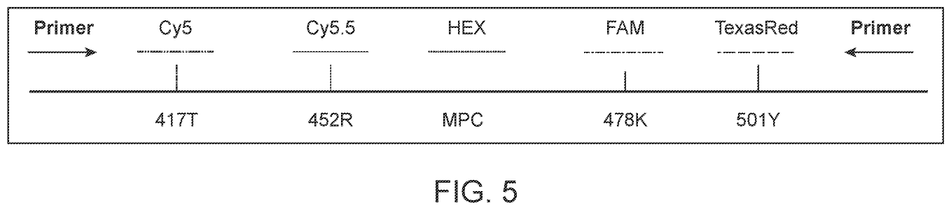

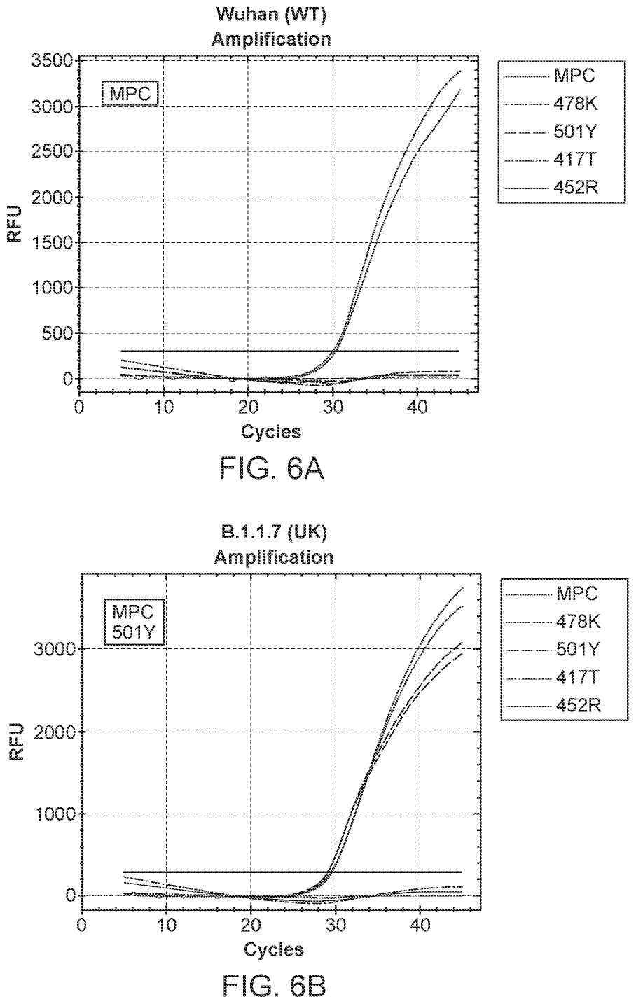

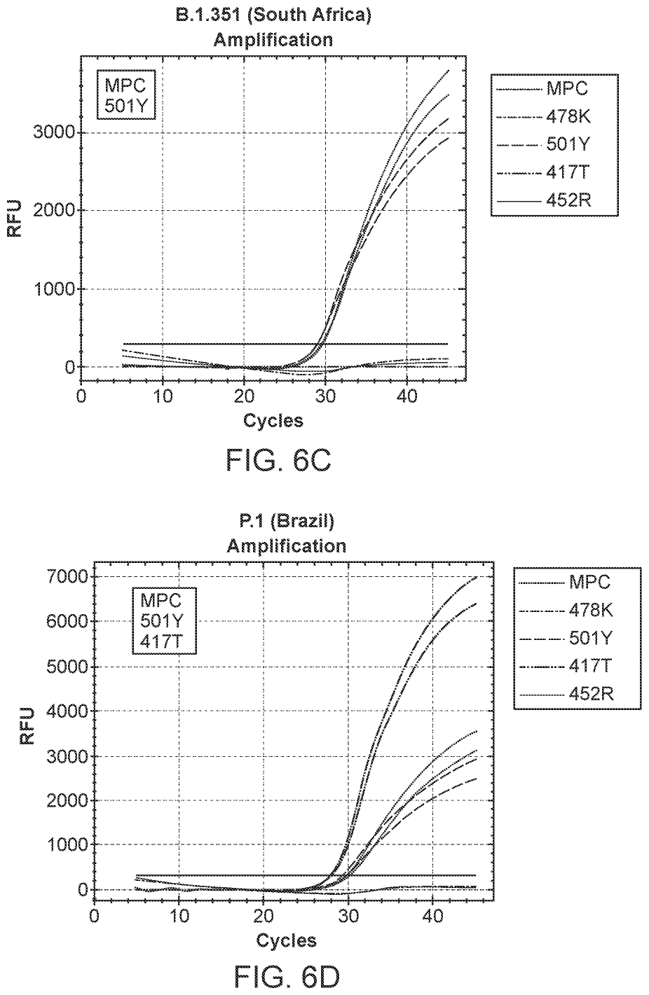

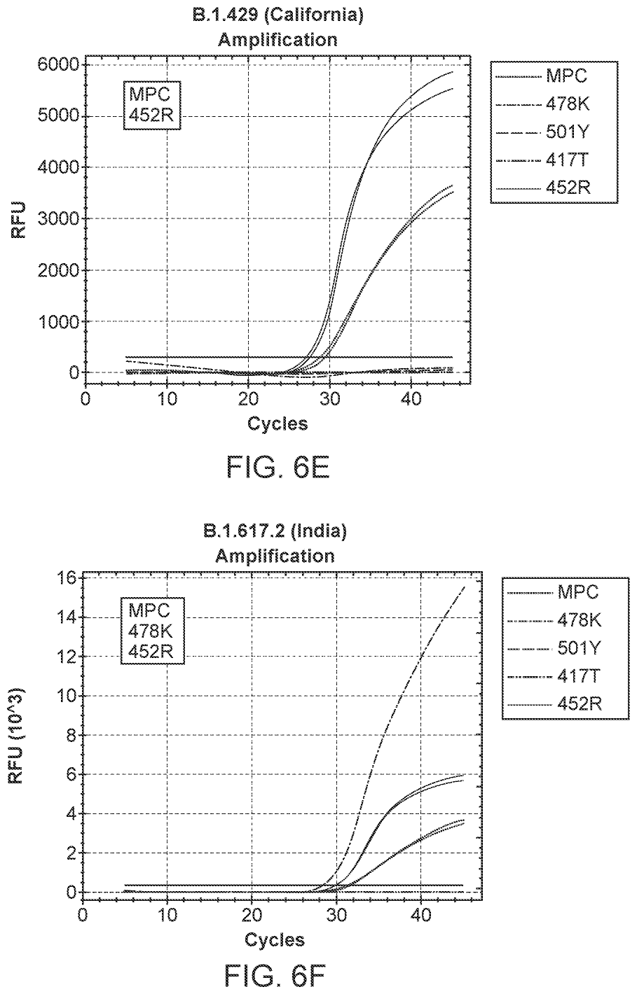

A second series of probes can be used within the 330 bp amplicon region that contains the known key mutations of the SARS-CoV-2 spike protein RBD region. 5 dual-labeled probes are designed to detect (1) a highly conserved region that serves as the control (HEX channel), (2) the 417T mutation (Cy5 channel), (3) the 452R mutation (Cy5.5 channel), (4) the 478K mutation (FAM channel), and (5) 501Y mutation (Texas Red) channel (FIG. 5). The final reaction mix contains the two primers and the 5 probes added to the Reliance One-Step Multiplex Supermix and RNA extracted from respiratory patient samples. Positive amplification in the control channel (HEX) indicates that the samples contain SARS-CoV-2 virus. Depending on which mutation is amplified, the variant strain can be determined. The Brazil variant can be identified if 501Y and 417T amplify (FIG. 6D). The California variant can be identified if 452R amplifies (FIG. 6E). The India variant can be identified if 478K and 452R amplify (FIG. 6F). If the sample contains the wild-type SARS-CoV-2, only the positive control is expected to be amplified (FIG. 6A).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A method for the amplification and detection of at least one genetic variation or at least two different genetic variations in a target nucleic acid sequence of about 60 bp to about 5000 bp from a sample, comprising:
a) optionally, isolating the nucleic acid sequence from the sample;
b) optionally, reverse transcribing the nucleic acid sequence to produce a cDNA sequence;
c) submitting said sample comprising a nucleic acid sequence comprising said target nucleic acid sequence or a nucleic acid extracted thereof or cDNA sequence derived from the nucleic acid sequence of the sample to nucleic acid amplification using a pair of oligonucleotide primers that amplify a portion of the sample nucleic acid sequence that is about 60 bp to about 5000 base pairs (bp) in length (the target nucleic acid sequence) and a plurality of fluorescent oligonucleotide probes, wherein each of the probes target a distinct genetic variation within the about 60 bp to about 5000 bp target nucleic acid sequence and the target nucleic acid sequence contains at least one genetic variation; and
d) detecting the fluorescence of each fluorescent oligonucleotide probe that anneals to the sample target nucleic acid sequence.

2. The method of claim 1, wherein the plurality of oligonucleotide fluorescent probes is between about 2 and about 125 or about 3 to about 6 distinct fluorescent oligonucleotide probes.

3. The method of claim 1, wherein the target nucleic acid sequence is about 60 bp to about 5000 bp, about 60 bp to about 4000 bp, about 60 bp to about 3000 bp, about 60 bp to about 2000 bp, about 60 bp to about 1000 bp, about 60 bp to about 750 bp, about 60 bp to about 500 bp, about 100 bp to about 500 bp, about 100 bp to about 750 bp, about 100 to about 1000 bp, about 100 bp to about 2000 bp, about 100 bp to about 3000 bp, about 100 bp to about 4000 bp, or about 100 bp to about 5000 bp.

4. The method of claim 1, further comprising submitting said sample comprising a nucleic acid sequence comprising said target nucleic acid sequence or a nucleic acid extracted thereof or cDNA sequence derived from the nucleic acid sequence of the sample to at least one DNA polymerase with 5′-3′ exonuclease activity, at least one dNTP, and at least one buffer having a pH adapted to the polymerase activity of said at least one DNA polymerase.

5. The method of claim 1, wherein at least one fluorescent oligonucleotide probe targets a constant region within the about 60 bp to about 5000 bp target nucleic acid sequence.

6. The method of claim 5, further comprising:
a) submitting said sample comprising a nucleic acid sequence comprising said target nucleic acid sequence or a nucleic acid extracted thereof to at least one reverse transcriptase, at least one DNA polymerase with 5′-3′ exonuclease activity, at least one dNTP, and at least one buffer having a pH adapted to the polymerase activity of said at least one DNA polymerase and said reverse transcriptase;
b) reverse transcribing the nucleic acid sequence comprising said target nucleic acid sequence or a nucleic acid extracted thereof, wherein a cDNA nucleic acid sequence is synthesized;
c) amplifying the cDNA nucleic acid sequence; and
d) detecting the at least distinct genetic variation and the at least one constant region.

7. The method of claim 1, wherein the about 60 bp to about 5000 bp target nucleic acid sequence comprises at least one single nucleotide polymorphism or encodes:
a) a SARS-CoV spike protein; or
b) an antigenic determinant of an infectious agent.

8. The method of claim 1, submitting said sample comprising a nucleic acid sequence comprising said target nucleic acid sequence or a nucleic acid extracted thereof or cDNA sequence derived from the nucleic acid sequence of the sample to 5 fluorescent oligonucleotide probes, wherein the fluorescent oligonucleotide probes comprise SEQ ID NO: 3, SEQ ID NO: 4, SEQ ID NO: 5, SEQ ID NO: 6, and SEQ ID NO: 7, and the pair of primers comprise SEQ ID NO: 1 and SEQ ID NO: 2.

9. The method of claim 1, submitting said sample comprising a nucleic acid sequence comprising said target nucleic acid sequence or a nucleic acid extracted thereof or cDNA sequence derived from the nucleic acid sequence of the sample to 5 fluorescent oligonucleotide probes, wherein the fluorescent oligonucleotide probes comprise SEQ ID NO: 8, SEQ ID NO: 4, SEQ ID NO: 5, SEQ ID NO: 9, and SEQ ID NO: 7, and the pair of primers comprise SEQ ID NO: 1 and SEQ ID NO: 2.

10. The method of claim 1, further comprising detecting the at least one genetic variation or at least two different genetic variations from two or more distinct biological samples.

11. The method of claim 1, wherein the target nucleic acid sequence is viral, bacterial, eukaryotic, or from a diseased cell or tissue.

12. The method of claim 1, wherein the target nucleic acid sequence contains one or more genetic variations.

13. The method of claim 1, wherein the target nucleic acid sequence contains two or more genetic variations.

14. The method of claim 1, wherein each of the fluorescent probes have a distinct fluorescent signal.

15. The method of claim 1, said method comprising amplification of one or more nucleic acid sequences using a pair of oligonucleotides primers that amplify a portion of nucleic acid sequences that are selected from qualitative controls, positive controls, negative controls, internal controls, quantitative controls, internal quantitative controls, or combinations thereof, the oligonucleotide primer pair optionally amplifying the target nucleic acid sequence and a positive control sequence within the target nucleic acid sequence.

16. The method of claim 15, wherein:
a) the positive control for a sample from a SARS-CoV-2 subject is selected from portions of the envelope gene, membrane gene, nucleocapsid gene, or invariant (unmutated) regions of the gene encoding the spike protein and can, optionally, be amplified by the same primers used to amplify the target nucleic acid sequence containing mutations in the RBD portion of the spike protein; or
b) the positive control for a tissue sample from a human subject is selected from the beta-actin gene, the aldolase gene, the dihydrofolate reductase gene, the glyceraldehyde phosphate dehydrogenase gene, the histone 3.3 gene, the hypoxanthine phosphoribosyltransferase gene, the Abelson gene (ABL), the BCR gene, the porphobilinogen deaminase gene (PBGD), or the beta-2-microglobulin gene (β2-MG).

17. A set of oligonucleotides, wherein:
a) the set of oligonucleotides comprises a pair of primers that amplify nucleic acid sequence of about 100 bp to about 1000 bp and 2, 3, 4, 5, 6, 7, 8, 9, or 10 fluorescent oligonucleotide probes, wherein each of the probes target a distinct portion of the about 100 bp to about 1000 bp nucleic acid sequence and each of the probes has a distinct fluorophore and, optionally, pairs of oligonucleotides primers that amplify a portion of nucleic acid sequences that are selected from qualitative controls, positive controls, negative controls, internal controls, quantitative controls, internal quantitative controls, or combinations thereof and probes that detect said qualitative controls, positive controls, negative controls, internal controls, quantitative controls, internal quantitative controls, or combinations thereof, each of said probes comprising a distinct fluorophore;
b) the set of oligonucleotides comprises an oligonucleotide comprising SEQ ID NO: 3, an oligonucleotide comprising SEQ ID NO: 4, an oligonucleotide comprising SEQ ID NO: 5, an oligonucleotide comprising SEQ ID NO: 6, and an oligonucleotide comprising SEQ ID NO: 7; or
c) wherein the set of oligonucleotides comprises an oligonucleotide comprising SEQ ID NO: 8, an oligonucleotide comprising SEQ ID NO: 4, an oligonucleotide comprising SEQ ID NO: 5, an oligonucleotide comprising SEQ ID NO: 9, and an oligonucleotide comprising SEQ ID NO: 7.

18. The set of oligonucleotides of claim 17, further comprising an oligonucleotide comprising SEQ ID NO: 1 and an oligonucleotide comprising SEQ ID NO: 2.

19. An amplification mix, which comprises:
at least one set of oligonucleotides of claim 17.

20. A kit, which comprises:
at least one set of oligonucleotides of claim 17.

21. The kit of claim 20, which further comprises at least one of the following elements:
at least one DNA polymerase;
at least one dNTP; or
at least one buffer having a pH adapted to the polymerase activity of said at least one DNA polymerase.


SEQUENCE LISTING (9 sequences)

SEQ ID NO: 1 - DNA, 25 bp, synthetic construct
  source 1..25: mol_type = other DNA
tcatttgtaattagaggtgatgaag

SEQ ID NO: 2 - DNA, 25 bp, synthetic construct
  source 1..25: mol_type = other DNA
agttcaaaagaaagtactactactc

SEQ ID NO: 3 - DNA, 25 bp, synthetic construct
  source 1..25: mol_type = other DNA
cttgtaatggtgttmaaggttttaa

SEQ ID NO: 4 - DNA, 25 bp, synthetic construct
  source 1..25: mol_type = other DNA
ccatatgattgtaaaggaaagtaac

SEQ ID NO: 5 - DNA, 18 bp, synthetic construct
  source 1..18: mol_type = other DNA
ccaacccacttatggtgt

SEQ ID NO: 6 - DNA, 22 bp, synthetic construct
  source 1..22: mol_type = other DNA
caaactggaaatattgctgatt

SEQ ID NO: 7 - DNA, 23 bp, synthetic construct
  source 1..23: mol_type = other DNA
attaccggtatagattgtttagg

SEQ ID NO: 8 - DNA, 18 bp, synthetic construct
  source 1..18: mol_type = other DNA
ccggtagcaaaccttgta

SEQ ID NO: 9 - DNA, 18 bp, synthetic construct
  source 1..18: mol_type = other DNA
tcagcaatcgttccagtt